(19) United States
(12) Patent Application Publication
(10) Pub. No.: US 2025/0261706 A1
(43) Pub. Date: Aug. 21, 2025

(54) COMPRESSION BRA FOR DECONGESTION CARE

(71) Applicant: THUASNE DEUTSCHLAND GMBH, Großburgwedel (DE)

(72) Inventors: Petra SCHÜTTE-BRUNS, Bissendorf (DE); Ina HEROLD, Chemnitz (DE)

(21) Appl. No.: 18/864,405

(22) PCT Filed: Apr. 17, 2023
(86) PCT No.: PCT/EP2023/059860

§ 371 Date: Nov. 8, 2024

(30) Foreign Application Priority Data
May 9, 2022 (DE) 10 2022 111 479.1

(51) Int. Cl. A41C 3/00

(52) U.S. Cl. CPC A41C 3/0035; A41C 3/0064

(57) ABSTRACT

A compression bra for decongestion care of the breast area includes an underband and two cups arranged above the underband and possibly accommodating breast pads. The compression bra includes at least one means for accommodating a lymphatic drainage pad. The accommodating means may configured as laterally open that extend up into the back area of the compression bra.

### FIELD OF DISCLOSURE

The invention relates to a compression bra for decongestion care, comprising an underbust portion and two cups arranged above the underbust portion for the possible accommodating of breast prostheses.

### BACKGROUND OF THE INVENTION

Such compression bras are basically known. For example, such a compression bra is described in DE 20 2008 001 456 U1.

Depending on the type of operation, therefore also after a breast-conserving operation, breast prostheses are inserted. In the case of operations with distinct volume deficits of the breasts or after removal of the breast, breast prostheses are typically inserted. This is mostly not necessary in the case of breast-conserving operations.

Secondary lymphoedema constitutes the most frequent long-term complication after breast cancer therapy. Acquired (secondary) lymphoedema occurs distinctly more frequently than congenital (primary) lymphoedema, in which the lymphatic pathways are not laid out correctly. Approximately 20-30% of breast cancer patients are affected by secondary lymphoedemas of the arms with/without breast and/or thorax wall. The lymphoedemas mostly occur only weeks or months after a cancer operation. On average, the lymphoedema occurs after ca. 15 months.

The first symptoms of a lymphoedema can be heaviness, tightness, pain and an incipient swelling. Once a lymphoedema has formed, it does not go away again on its own, but steadily worsens. If the lymphoedema is not treated, the affected part of the body increases in circumference, the tissue changes. The swelling, which can initially still be pressed in and moved, becomes hard. People who are affected require urgently a so-called complex physical decongestion therapy (CPDT). This therapy programme consists, inter alia, of the components:
manual lymph drainage
compression treatment with bandages, compression stockings and compression bras

A secondary lymphoedema requires a permanent therapy. This means a lifelong wearing of the compression aids and, if applicable, the carrying out of manual maintenance lymphatic drainages.

Generally, even in the case of therapy which is carried out consistently, only the worsening of the clinical picture is prevented.

### SUMMARY

The invention is therefore based on the problem of creating a compression bra of the type in the introduction, which is able to be coordinated optimally and flexibly to the respective oedema condition of the patient.

The solution to this problem takes place with a compression bra having the features of claim 1. Advantageous embodiments of the invention are described in the subclaims.

In the case of a compression bra for decongestion care comprising an underbust band and two cups arranged above the underbust band for possibly accommodating breast prostheses, provision is made in a manner essential to the invention that the compression bra has at least one accommodating means for accommodating a lymphatic drainage pad. With such an accommodating means, in which a lymphatic drainage pad can be accommodated, which can be replaced accordingly, the compression bra is able to be adapted in a flexible manner. By only the lymphatic drainage pad being replaced, therefore by exchanging the lymphatic drainage pad, typically from a larger lymphatic drainage pad to a smaller lymphatic drainage pad, the compression bra can be adapted to the respective oedema condition without great expenditure.

The compression bra can also be designated as a compression lymph bra. This serves for care and/or therapy after an operation. In particular, such a compression lymph bra is also to have a decongesting effect. The underbust band can also be configured over a larger area and can then be rather considered as an underbust portion. It is essential that an area is present here beneath the cups, which is tight-fitting.

Preferably, the compression bra has two accommodating means for accommodating lymphatic drainage pads. Preferably here an accommodating means for accommodating a lymphatic drainage pad is associated with each cup.

In a further preferred embodiment of the invention, the accommodating means are configured as open pockets into which the lymphatic drainage pads can be inserted. Preferably, these are configured such that the pockets are open laterally, so that the lymphatic drainage pads can be inserted laterally into the pockets.

In a particularly preferred embodiment of the invention, the pockets are arranged up into the back area of the compression bra. The decongesting pressure of the compression bra can therefore be exerted up into the back area. Particularly preferably, the pockets extend from approximately the mid-height of the shoulder blade up to the underbust band, and extend under the armpit into the vicinity of the cups.

In another preferred embodiment of the invention, the lymphatic drainage pads have approximately an “L”-shape. This shape is adapted to the pockets, wherein preferably the pockets have a corresponding shape into which the lymphatic drainage pads are able to be inserted.

In this preferred embodiment of the invention, the lymphatic drainage pads are formed from a structured material which has elevations and depressions. Thereby, an airflow and a care of the skin are ensured. This is preferably achieved in that the lymphatic drainage pads are formed from a plurality of foam cubes which are held by an upper and a lower layer of a material. This constitutes a preferred embodiment, by which the lymphatic drainage pad can be produced with elevations and depressions, which enable an air permeability to a plurality of selective pressure differences. The material is preferably a non-woven fabric.

The irregular structure of the lymphatic drainage pads increases the decongesting effect through the resulting pressure differences in the fabric.

In another further development of the invention, several lymphatic drainage pads are inserted over one another, or respectively the compression bra is configured such that several lymphatic drainage pads are able to be inserted over one another.

The compression bra is preferably configured so that the back portion 12 extends 12 cm to 16 cm to below the cervical vertebra C7.

Furthermore, a lymphatic drainage pad is claimed for use in a compression bra according to one of the preceding claims. The lymphatic drainage pad described in claims 7 to 11 or respectively the lymphatic drainage pad described in the above paragraphs is part of the invention as an individual part or replacement part for use with the compression bra.

### BRIEF DESCRIPTION OF THE DRAWINGS

The invention is further explained below with the aid of an example embodiment, illustrated in the drawings. In detail, the schematic illustrations show in:

FIG. 1: a compression bra with the inner side for illustration outwards and breast prosthesis inserted to the right;

FIG. 2: a compression bra in back view with the inner side for illustration outwards and inserted lymphatic drainage pads;

FIG. 3: a compression bra in side view with the inner side for illustration outwards and inserted lymphatic drainage pads;

FIG. 4: a compression bra in front view, flat;

FIG. 5: a compression bra in front view, flat, with the inner side upwards with inserted lymphatic drainage pads;

FIG. 6: a compression bra in front view, flat, with the inner side upwards with applied lymphatic drainage pads; and

FIG. 7: a lymphatic drainage pad for compression bra right/left with foam cubes between the non-woven fabrics.

### DETAILED DESCRIPTION

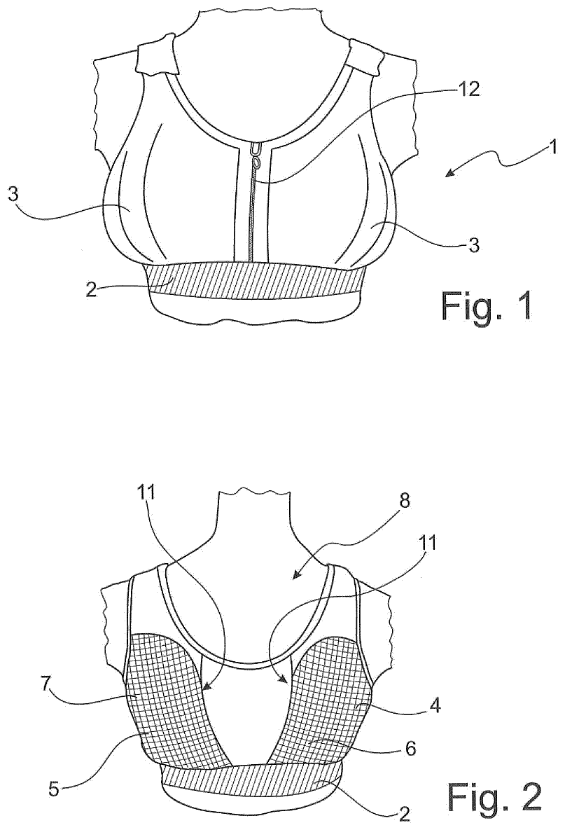

In FIG. 1 a compression bra 1 is illustrated, which has two cups 3, beneath which an underbust band 2 is provided which positions the compression bra in a secure and reliable manner. Between the two cups 3 a vertical zip fastener 12 is provided, by which the compression bra 1 can be opened.

In FIG. 2 the compression bra 1 is illustrated in a back view. There, above the underbust band 2, accommodating means 4 and 5 are provided, into which lymphatic drainage pads 6 and 7 are inserted. The lymphatic drainage pads 6 and 7 are inserted into this back area 8 and thereby provide as a whole for a compression or exertion of pressure in the entire area of the compression bra 1. The accommodating means 4, 5 are configured as laterally open pockets 11, so that the lymphatic drainage pads 6, 7 can be inserted into these pockets 11, which are aligned here to the middle of the body.

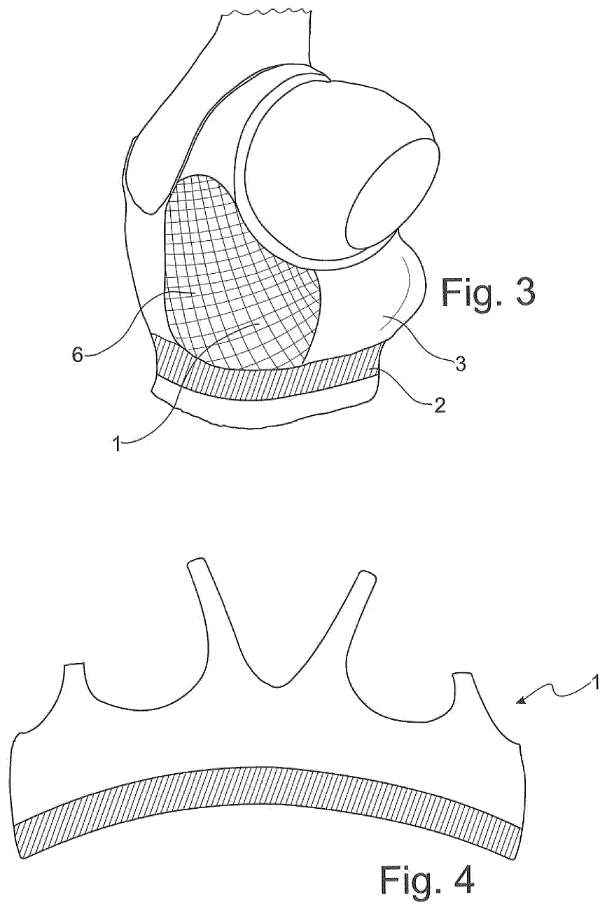

In FIG. 3 the compression bra 1 is illustrated with the underbust band 2 in a side view. Here, also, the lymphatic drainage pad 6 and the associated cup 3 can be readily seen. A pressure onto the cup 3 is exerted by the lymphatic drainage pad 6.

In FIG. 4 the compression bra 1 is illustrated alone, therefore without the body. In FIG. 4 the compression bra 1 is illustrated in front view.

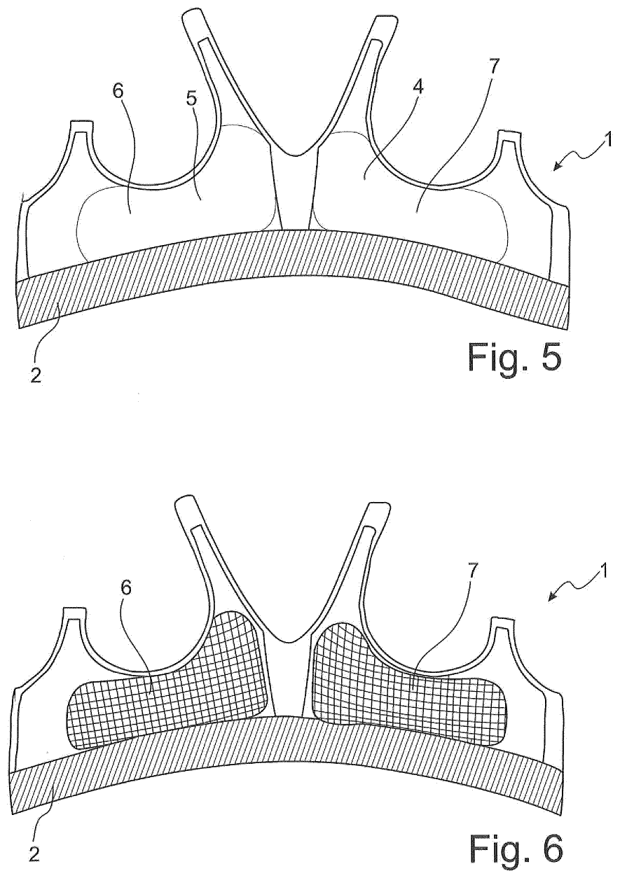

FIG. 5 shows the compression bra 1 inside out, with inserted lymphatic drainage pads 6 and 7.

FIG. 6 shows the compression bra 1 inside out with applied lymphatic drainage pads 6 and 7. The lymphatic drainage pads 6, 7 here are therefore not, as in FIG. 5, inserted into the accommodating means 4, 5, but rather applied for clarification. It can also be seen here that the lymphatic drainage pads 6, 7 are structured.

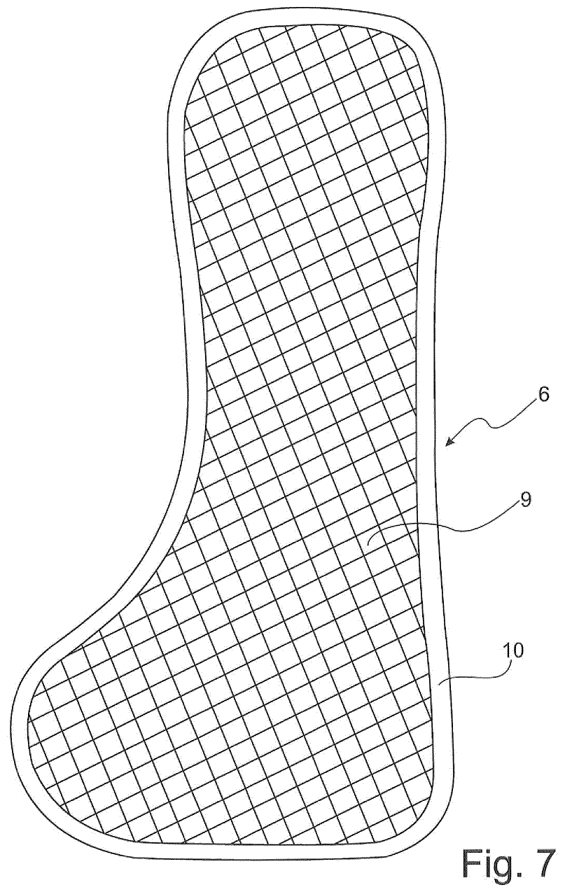

In FIG. 7 a lymphatic drainage pad 6 is illustrated alone. It can be seen here that the lymphatic drainage pad 6 consists of a plurality of foam cubes 9, which provide for the lymphatic drainage pad 6 to be structured. The foam cubes 9 are held between two non-woven fabrics or material layers 10.

1. A compression bra for decongestion care of the breast area, comprising an underbust band and two cups arranged above the underbust band for the possible accommodating of breast prostheses,
wherein the compression bra comprises at least one accommodating means for accommodating a lymphatic drainage pad.

2. The compression bra according to claim 1, wherein the compression bra comprises two accommodating means for accommodating lymphatic drainage pads.

3. The compression bra according to claim 1, wherein the accommodating means are configured as open pockets.

4. The compression bra according to claim 3, wherein the accommodating means are configured as laterally open pockets.

5. The compression bra according to claim 3, wherein the pocket extends up into the back area of the compression bra.

6. The compression bra according to claim 5, wherein the pockets extend from approximately the mid-height of the shoulder blade to the underbust band and extend under the armpit to above the cups.

7. The compression bra according to claim 1, wherein the lymphatic drainage pads have approximately an “L-shape”.

8. The compression bra according to claim 1, wherein the lymphatic drainage pads are formed from a structured material, which comprises elevations and depressions.

9. The compression bra according to claim 1, wherein the lymphatic drainage pads are formed from a plurality of foam cubes, which are held by an upper and a lower layer of a material.

10. The compression bra according to claim 9, wherein the foam cubes have an edge length of 3-15 mm, in particular 4-6 mm.

11. The compression bra according to claim 1, wherein several lymphatic drainage pads are able to be inserted over one another.

12. The compression bra according to claim 1, wherein the lymphatic pads are cut to size individually with scissors.

13. The compression bra according to claim 1, wherein the back portion extends 12 cm to 16 cm below a cervical vertebra.

14. A lymphatic drainage pad for use in the compression bra according to claim 1.
